(19) United States
(12) Patent Application Publication
(10) Pub. No.: US 2023/0226156 A1
(43) Pub. Date: Jul. 20, 2023

(54) COMPOSITE NANOMATERIAL BASED ON METAL-ORGANIC FRAMEWORK MATERIAL LOADED WITH HORSERADISH PEROXIDASE AND PREPARATION METHOD AND USE THEREOF

(71) Applicant: LINYI UNIVERSITY, Linyi City (CN)

(72) Inventors: Pengfei SHI, Linyi City (CN); Guoda ZHANG, Linyi City (CN); Yingyan LIU, Linyi City (CN); Shusheng ZHANG, Linyi City (CN)

(21) Appl. No.: 17/823,715

(22) Filed: Aug. 31, 2022

(30) Foreign Application Priority Data
Jan. 17, 2022 (CN) 202210048000.3

(51) Int. Cl. A61K 38/44; C12N 11/02; C12N 9/08; A61K 47/69; A61P 35/00

(52) U.S. Cl. CPC A61K 38/44; C12N 11/02; C12N 9/0065; A61K 47/6921; A61P 35/00

(57) ABSTRACT

Disclosed are a composite nanomaterial based on a metal-organic framework (MOF) material loaded with horseradish peroxidase (HRP) and a preparation method and use thereof. The composite nanomaterial based on the MOF material loaded with HRP includes a hafnium-based MOF material and HRP loaded thereon, where the hafnium-based MOF material is formed by self-assembly of 2′-amino-1,1′:4,1″-terphenyl-4,4″-dicarboxylic acid and hafnium ions through a coordination bond.

### CROSS REFERENCE TO RELATED APPLICATION

This patent application claims the benefit and priority of Chinese Patent Application No. 202210048000.3, entitled “Composite nanomaterial based on metal-organic framework material loaded with horseradish peroxidase and preparation method and use thereof” filed on Jan. 17, 2022, the disclosure of which is incorporated by reference herein in its entirety as part of the present application.

### TECHNICAL FIELD

The present disclosure relates to the technical field of functional materials, in particular to a composite nanomaterial based on a metal-organic framework (MOF) material loaded with horseradish peroxidase (HRP) and a preparation method and use thereof.

### BACKGROUND ART

An enzyme, as an efficient and green biomacromolecular catalyst composed of linear amino acid chains, has a foldable, complex and precise tertiary structure. Without changing the reaction equilibrium and without consuming the enzyme, the enzyme can greatly improve reaction efficiency by reducing an activation energy. In catalytic applications, the enzyme suffers from poor stability, which limits effectiveness and longevity of the enzyme in complex applications.

Metal-organic frameworks (MOFs) are organic-inorganic hybrid materials with intramolecular pores formed by self-assembly of organic ligands and metal ions or clusters through coordination bonds. The MOFs have become one of the most interesting materials for researchers due to an ultra-high specific surface area, porosity, stability, and tunable size thereof.

The immobilization of enzymes with MOFs as carriers could improve stability of free enzymes. For example, it has been reported that HRP or glucose oxidase was loaded on ZIF-8. However, ZIF-8 is easily destroyed due to poor stability in the tumor cell microenvironment, losing the protective function of enzymes.

### SUMMARY

An object of the present disclosure is to provide a composite nanomaterial based on an MOF material loaded with HRP and a preparation method and use thereof. In the present disclosure, the hafnium-based MOF material with a relatively desirable stability in an acidic environment is used as a carrier to load HRP, which makes it possible to protect HRP from being decomposed, and meanwhile improve the catalytic ability of HRP, thereby achieving tumor treatment.

To achieve the above object, the present disclosure provides the following technical solutions.

The present disclosure provides a composite nanomaterial based on an MOF material loaded with HRP, including a hafnium-based MOF material and HRP loaded thereon, wherein the hafnium-based MOF material is formed by self-assembly of 2′-amino-1,1′:4,1″-terphenyl-4,4″-dicarboxylic acid and hafnium ions through a coordination bond.

In some embodiments, the composite nanomaterial based on an MOF material loaded with HRP has a loading amount of HRP of 11 wt % to 12 wt %.

In some embodiments, the hafnium-based MOF material has a particle size of 500 nm to 550 nm.

The present disclosure further provides a method for preparing the composite nanomaterial based on an MOF material loaded with HRP as described in the above technical solutions, including the following steps:

providing the hafnium-based MOF material, wherein the hafnium-based MOF material is formed by self-assembly of 2′-amino-1,1′:4,1″-terphenyl-4,4″-dicarboxylic acid and the hafnium ions through the coordination bond; and

mixing the hafnium-based MOF material, HRP, and water to obtain a first mixture, and subjecting the first mixture to an incubation to obtain the composite nanomaterial based on an MOF material loaded with HRP.

In some embodiments, each of the hafnium-based MOF material and HRP in the mixture independently has a concentration of 9 mg/mL to 11 mg/mL.

In some embodiments, the incubation is conducted at a temperature of 35° C. to 39° C. for 25 min to 35 min.

In some embodiments, the hafnium-based MOF material is prepared by a process including the following steps:

mixing hafnium tetrachloride, 2′-amino-1,1′:4,1″-terphenyl-4,4″-dicarboxylic acid, N,N′-dimethylformamide and acetic acid to obtain a second mixture; and

subjecting the second mixture to a solvothermal reaction to obtain the hafnium-based MOF material.

The present disclosure further provides use of the composite nanomaterial based on an MOF material loaded with HRP as described in the above technical solutions or a composite nanomaterial based on an MOF material loaded with HRP prepared by the method as described in the above technical solutions in preparation of a drug for inhibiting growth of tumor cells.

In some embodiments, the tumor cells include lung adenocarcinoma cells and/or cervical cancer cells.

The present disclosure provides a composite nanomaterial based on an MOF material loaded with HRP, including a hafnium-based MOF material and HRP loaded thereon, wherein the hafnium-based MOF material is formed by self-assembly of 2′-amino-1,1′:4,1″-terphenyl-4,4″-dicarboxylic acid and hafnium ions through a coordination bond. In the present disclosure, the hafnium-based MOF material (Hf-DBA) with a relatively desirable stability in an acidic environment is used as a carrier to load HRP, which makes it possible to protect HRP from being decomposed, and meanwhile improve a catalytic ability of HRP, such that HRP could play a catalytic role in a tumor microenvironment to generate reactive oxygen species to kill tumor cells, thereby achieving tumor treatment.

The present disclosure further provides a method for preparing the composite nanomaterial based on an MOF material loaded with HRP (HRP@Hf-DBA) as described above. In the present disclosure, the method has simple operation and desirable safety; the prepared composite nanomaterial based on an MOF material loaded with HRP has desirable stability and could maintain structural integrity in an acidic tumor microenvironment; on this basis, HRP exhibits enhanced catalytic performance, thus achieving tumor treatment.

Moreover, in the present disclosure, the hafnium-based MOF material is prepared by a solvothermal method, with a simple operation.

### BRIEF DESCRIPTION OF THE DRAWINGS

FIG. 1 shows a transmission electron microscope (TEM) image of the Hf-DBA.

FIG. 2 shows X-ray diffraction (XRD) patterns of the Hf-DBA and the HRP@Hf-DBA.

FIG. 3 shows UV absorption spectra of the Hf-DBA and the HRP@Hf-DBA.

FIGS. 4A-4D show enzymatic catalytic performance of the HRP@Hf-DBA under different concentrations of 3,3′,5,5′-tetramethylbenzidine (TMB), H₂O₂, and HRP@Hf-DBA and different pH values in Use Example 1.

FIG. 5A shows a comparison of enzymatic activities of HRP and the HRP@Hf-DBA at different pH in Use Example 1.

FIG. 5B shows a comparison of enzymatic activities of HRP and the HRP@Hf-DBA at different temperatures in Use Example 1.

FIG. 5C shows enzymatic activities of the HRP@Hf-DBA at different cycles in Use Example 1.

FIG. 6 shows fluorescence emission spectra of 2′,7′-dichlorofluorescein (DCF) under different grouping conditions in Use Example 2.

FIG. 7A shows results of a cytotoxicity test-of tumor cells at different concentration of Hf-DBA in Use Example 3.

FIG. 7B shows results of a cytotoxicity test of tumor cells for the complete culture substrate, Hf-DBA, and HRP@Hf-DBA in Use Example 3.

FIG. 7C shows results of confocal imaging of tumor cell A549 in Use Example 3.

FIG. 7D shows results of confocal imaging of tumor cell Hela in Use Example 3.

FIG. 8 shows results of apoptosis of tumor cells in Use Example 3.

### DETAILED DESCRIPTION OF THE EMBODIMENTS

The present disclosure provides a composite nanomaterial based on an MOF material loaded with HRP, including a hafnium-based MOF material and HRP loaded thereon, wherein the hafnium-based MOF material is formed by self-assembly of 2′-amino-1,1′:4,1″-terphenyl-4,4″-dicarboxylic acid and hafnium ions through a coordination bond. Generally, the tumor microenvironment is acidic, which will decompose HRP and reduce the catalytic performance thereof. In the present disclosure, the hafnium-based MOF material is used as a carrier to load HRP. The hafnium-based MOF material is a porous material, which has desirable stability in the acidic environment of tumors and could still maintain a desirable pore structure. In some embodiments, HRP is specifically loaded in pores of the hafnium-based MOF material to prevent it from being decomposed; accordingly, HRP could better catalyze the Fenton reaction with hydrogen peroxide rich in tumor cells to generate reactive oxygen species, thus treating tumors by chemodynamic therapy.

In some embodiments, the hafnium-based MOF material has a particle size of 500 nm to 550 nm, preferably 500 nm to 520 nm. In some embodiments, the composite nanomaterial based on an MOF material loaded with HRP has a loading amount of HRP of 11 wt % to 12 wt %, preferably 11.35 wt %.

The present disclosure further provides a method for preparing the composite nanomaterial based on an MOF material loaded with HRP as described in the above technical solutions, including the following steps:

providing the hafnium-based MOF material, wherein the hafnium-based MOF material is formed by self-assembly of 2′-amino-1,1′:4,1″-terphenyl-4,4″-dicarboxylic acid and hafnium ions through the coordination bond; and

mixing the hafnium-based MOF material, HRP, and water to obtain a first mixture, and subjecting the first mixture to an incubation to obtain the composite nanomaterial based on an MOF material loaded with HRP.

In the present disclosure, the hafnium-based MOF material is provided, wherein the hafnium-based MOF material is formed by self-assembly of 2′-amino-1,1′:4,1″-terphenyl-4,4″-dicarboxylic acid and hafnium ions through the coordination bond. In some embodiments, the hafnium-based MOF material is prepared by a process including the following steps:

mixing hafnium tetrachloride, 2′-amino-1,1′:4,1″-terphenyl-4,4″-dicarboxylic acid, N,N′-dimethylformamide and acetic acid to obtain a second mixture; and

subjecting the second mixture to a solvothermal reaction to obtain the hafnium-based MOF material.

In some embodiments, a mass ratio of hafnium tetrachloride to 2′-amino-1,1′:4,1″-terphenyl-4,4″-dicarboxylic acid is in a range of 1:(1.0-1.1), preferably 1:1. In some embodiments, a ratio of hafnium tetrachloride to N,N′-dimethylformamide to acetic acid is in a range of 0.0128 g:(3.1-3.3) mL:(0.23-0.25) mL, preferably 0.0128 g:3.2 mL:0.24 mL. In some embodiments, mixing hafnium tetrachloride, 2′-amino-1,1′:4,1″-terphenyl-4,4″-dicarboxylic acid, N,N′-dimethylformamide and acetic acid is performed by mixing hafnium tetrachloride and 2′-amino-1,1′:4,1″-terphenyl-4,4″-dicarboxylic acid to obtain a mixture I, adding N,N′-dimethylformamide thereto, and ultrasonically dispersing for 10 min to 20 min to obtain a mixture II, and adding acetic acid to the mixture II and mixing to be uniform. In some embodiments, the solvothermal reaction is conducted at a temperature of 115° C. to 125° C., preferably 120° C.; the solvothermal reaction is conducted for 70 h to 75 h, preferably 72 h. During the solvothermal reaction, 2′-amino-1,1′:4,1″-terphenyl-4,4″-dicarboxylic acid and hafnium ions are self-assembled through a coordination bond to form the hafnium-based MOF material. In some embodiments, after the solvothermal reaction, an obtained product system is cooled to ambient temperature and then subjected to a solid-liquid separation to obtain a solid, and the solid is washed with N,N′-dimethylformamide to obtain a crude product; and the crude product is washed with ethanol, and then left standing in ethanol to remove N,N′-dimethylformamide in pores of the crude product, to obtain the hafnium-based MOF material. There is no special limitation on the method of the solid-liquid separation, and methods well known to those skilled in the art may be used, such as centrifugation. In some embodiments, the standing is conducted in ethanol at a temperature of 55° C. to 65° C., preferably 60° C.; the standing is conducted for 1.5 days to 2.5 days, preferably 2 days. In some embodiments, during the standing, ethanol is replaced every 12 h. In some embodiments, after the standing, an obtained material is subjected to an ultrasonic washing with water to remove possible residual acetic acid in the material, to obtain the hafnium-based MOF material. In some embodiments, the ultrasonic washing is conducted 3 to 5 times for 15 min to 20 min in each time.

In the present disclosure, the hafnium-based MOF material, HRP, and water are mixed to obtain a first mixture, and the first mixture is subjected to an incubation to obtain the composite nanomaterial based on an MOF material loaded with HRP. In some embodiments, each of the hafnium-based MOF material and HRP in the mixture independently has a concentration of 9 mg/mL to 11 mg/mL, preferably 10 mg/mL. In some embodiments, the hafnium-based MOF material, HRP, and water are mixed by mixing the hafnium-based MOF material and HRP with water separately to obtain a hafnium-based MOF material aqueous dispersion and an HRP aqueous solution; and mixing the hafnium-based MOF material aqueous dispersion and the HRP aqueous solution. In some embodiments, the incubation is conducted at a temperature of 35° C. to 39° C., preferably 37° C.; the incubation is conducted for 25 min to 35 min, preferably 30 min. In some embodiments, after the incubation, an obtained product system is subjected to a solid-liquid separation to obtain a solid, and the solid is washed with water to obtain the composite nanomaterial based on an MOF material loaded with HRP. There is no special limitation on a method of the solid-liquid separation, and methods well known to those skilled in the art may be used, such as centrifugation.

The present disclosure further provides use of the composite nanomaterial based on an MOF material loaded with HRP as described in the above technical solutions or a composite nanomaterial based on an MOF material loaded with HRP prepared by the method as described in the above technical solutions in preparation of a drug for inhibiting growth of tumor cells. The composite nanomaterial based on an MOF material loaded with HRP specifically treats tumors through a chemodynamic therapy. In some embodiments, the tumor cells include lung adenocarcinoma cells and/or cervical cancer cells.

The technical solutions in the present disclosure will be clearly and completely described below in conjunction with examples of the present disclosure. It is clear that the described examples are merely a part, rather than all of the embodiments of the present disclosure. All other embodiments obtained by a person of ordinary skill in the art based on the examples of the present disclosure without creative efforts shall fall within the protection scope of the present disclosure.

### EXAMPLE 1

0.0128 g of hafnium tetrachloride (HfCl₄) and 0.0133 g of 2′-amino-1,1′:4,1″-terphenyl-4,4″-dicarboxylic acid (H₂DBA) were separately weighted and added into a 10 mL sample vial, and 3.2 mL of N,N′-dimethylformamide (DMF) was added thereto, and the resulting mixture was ultrasonically dispersed for 15 min, and 240 μL of acetic acid was added thereto and mixed to be uniform to obtain a mixed material. The sample vial containing the mixed material was put into an oven, and the mixed material was subjected to a reaction at 120° C. for 72 h. After the reaction, an obtained product system was cooled to ambient temperature (25° C.), and centrifuged to obtain a solid. The solid was washed three times with DMF to obtain a crude product. The crude product was washed 3 times with ethanol, and then left standing in ethanol at 60° C. for 2 days, wherein during the standing, ethanol was replaced every 12 h. An obtained material after the standing was subjected to an ultrasonic washing with water 3 times for 15 min in each time, to obtain a hafnium-based MOF material (Hf-DBA).

10 mg of the Hf-DBA was weighted and mixed with 1 mL of an HRP aqueous solution with a concentration of 10 mg/mL, and the resulting mixture was subjected to an incubation at 37° C. for 30 min. An obtained product system after the incubation was centrifuged to obtain a solid. The solid was washed 3 times with water to obtain a composite nanomaterial based on an MOF material loaded with HRP (HRP@Hf-DBA).

Characterization:

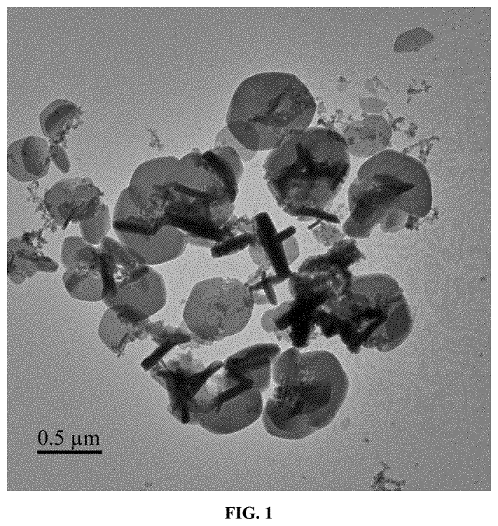

An appropriate amount of the Hf-DBA was dissolved in ethanol, and ultrasonically dispersed to obtain an Hf-DBA ethanol dispersion. A small amount of the Hf-DBA ethanol dispersion was added dropwise on a copper mesh as a test sample for characterization. Morphology and size of the Hf-DBA were characterized by a transmission electron microscope (TEM). The results are shown in FIG. 1, and the Hf-DBA has a particle size of about 500 nm.

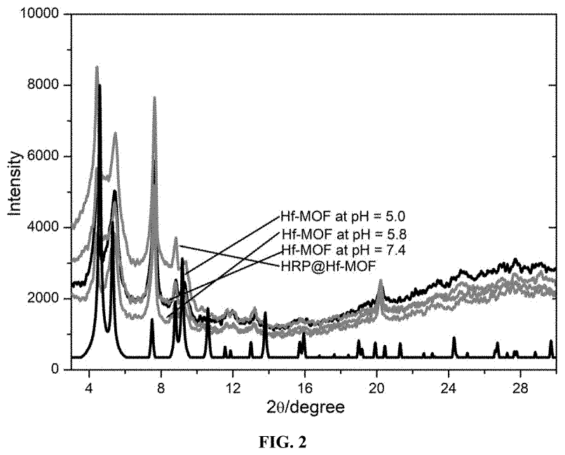

An equal amount of the Hf-DBA was dispersed in a PBS buffer solution with pH values of 5.0, 5.8 and 7.4 separately, and centrifuged after 12 h, and the obtained solids were dried. A crystal structure of the Hf-DBA was characterized by an X-ray diffractometer and compared with that of the HRP@Hf-DBA. The results are shown in FIG. 2. Compared with simulated diffraction peaks of a single crystal XRD, it can be seen that the Hf-DBA dispersed in PBS buffer solutions with different pH values for 12 h could keep the structure stable; compared with the simulated diffraction peaks of the single crystal XRD, the HRP@Hf-DBA does not change, indicating that the structure of Hf-DBA could still remain stable after being loaded with HRP.

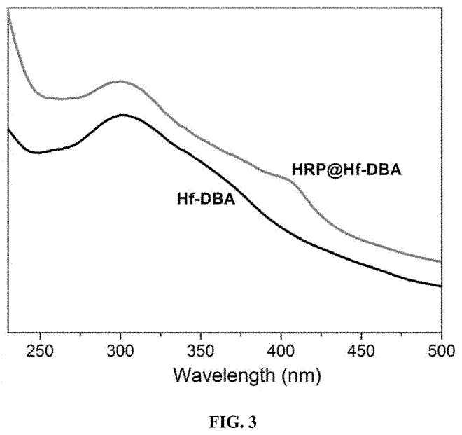

The Hf-DBA and HRP@Hf-DBA were subjected to an ultraviolet (UV) absorption test. The results are shown in FIG. 3. Compared with the Hf-DBA, the HRP@Hf-DBA exhibits a characteristic UV absorption peak of HPR at 403 nm, indicating that HRP has been fixed on the Hf-DBA.

Use Example 1 Enzymatic Catalytic Performance of HRP@Hf-DBA

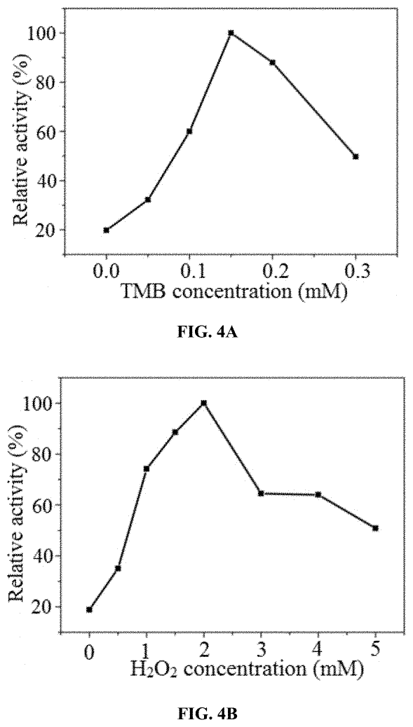

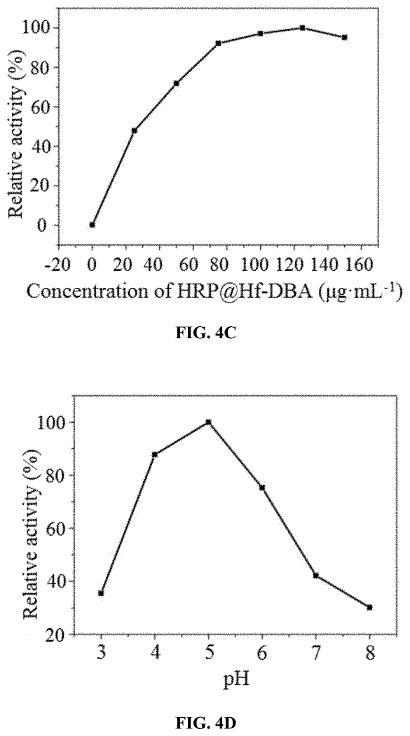

Peroxidase substrates can be detected by 3,3′,5,5′-tetramethylbenzidine (TMB). Specifically, peroxidase can catalyze generation of hydroxyl radicals (·OH) from H₂O₂, and ·OH can react with TMB to generate oxTMB showing blue, and absorption peaks can be measured at 370 nm and 652 nm. Therefore, in Use Example 1, TMB was selected as a chromogenic substrate, and a UV-Vis spectrophotometer was used to measure the change of the absorption peak at 370 nm. To study the enzymatic catalytic performance of HRP@Hf-DBA, factors affecting the enzymatic catalytic performance, i.e., TMB concentration (0, 0.05 mM, 0.1 mM, 0.15 mM, 0.2 mM, and 0.3 mM), H₂O₂ concentration (0, 0.5 mM, 1.0 mM, 1.5 mM, 2.0 mM, 3.0 mM, 4.0 mM, and 5.0 mM), HRP@Hf-DBA concentration (0, 25 μg/mL, 50 μg/mL, 75 μg/mL, 100 μg/mL, 125 μg/mL, and 150 μg/mL), and pH value (3, 4, 5, 6, 7, and 8), were optimized. The results are shown in FIGS. 4A-4D. Under the condition that the TMB concentration is 0.14 mM, the H₂O₂ concentration is 2 mM, the HRP@Hf-DBA concentration is 125 μg/mL, and the pH value is 5, the HRP@Hf-DBA has an optimal enzymatic catalytic activity.

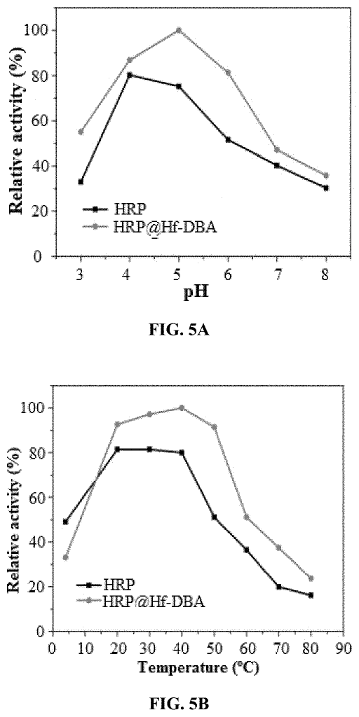

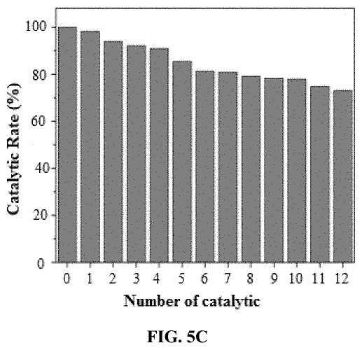

The protective effect of Hf-DBA on HRP was studied by changing the temperature or pH value, with other conditions being the above optimal conditions. Specifically, equal amounts of HRP@Hf-DBA and HRP (based on the amount of HRP) were weighed for enzyme activity test. As shown in FIGS. 5A and 5B, the enzymatic activity of HRP@Hf-DBA is better than that of free HRP, indicating that the Hf-DBA could protect HRP during the catalytic process and improve its enzymatic catalytic activity.

The catalytic stability of HRP@Hf-DBA was tested under the above optimal conditions. Specifically, HRP@Hf-DBA was added into a PBS buffer solution (pH=5) containing H₂O₂, and then a first catalytic reaction was conducted, wherein the HRP@Hf-DBA had a concentration of 125 μg/mL and H₂O₂ had a concentration of 2 mM. The reacted solution after the first catalytic reaction was centrifugated to obtain a solid. The obtained solid was washed 3 times with water, and then dispersed into 1 mL of the PBS buffer solution (pH=5) to obtain an HRP@Hf-DBA dispersion. The HRP@Hf-DBA dispersion was taken and tested. The above process was regarded as one catalytic cycle; the foregoing operations were repeated for 12 catalytic cycles in total. The results are shown in FIG. 5C. It can be seen that after 12 catalytic cycles, the catalytic rate of HRP@Hf-DBA remains not less than 70%; the decreased catalytic rate may be caused by washing away of HRP with weak binding of Hf-DBA and the loss of HRP@Hf-DBA during the washing. In addition, this result also proves that HRP is loaded on Hf-DBA and can maintain a strong catalytic rate.

Use Example 2 Detection of Reactive Oxygen Species Produced by HRP@Hf-DBA

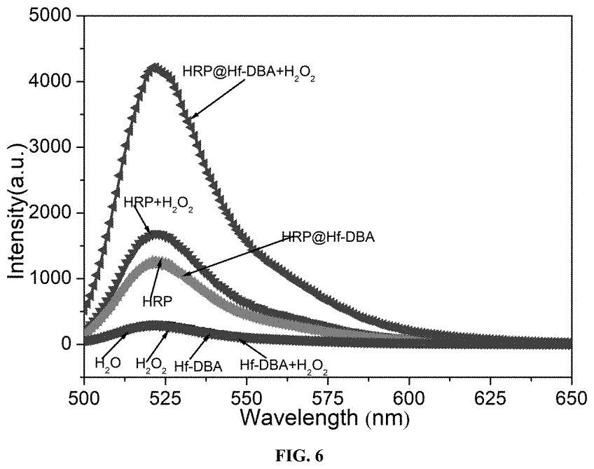

To study an ability of HRP@Hf-DBA to catalyze generation of reactive oxygen species (ROS) from H₂O₂, the ROS generated in vitro was detected by a 2′,7′-dichlorofluorescein diacetate (DCFH-DA) dye. The DCFH-DA did not have fluorescence itself, and could be oxidized into 2′,7′-dichlorofluorescein (DCF) with green fluorescence after reacting with the ROS. Specifically, a DCFH-DA aqueous solution, an aqueous dispersion of a material (i.e., HRP, Hf-DBA or HRP@Hf-DBA) and H₂O₂ were added into 1 mL of water according to grouping, such that the DCFH, the material and H₂O₂ had final concentrations of 10 mM, 125 μg/mL, and 2 mM, respectively. After 5 min, a fluorescence spectrophotometer was used to detect the fluorescence intensity of each group (Eₓ=488 nm, Eₘ=525 nm). The specific grouping was: H₂O, H₂O₂, HRP, HRP+H₂O₂, Hf-DBA, Hf-DBA+H₂O₂, HRP@Hf-DBA, and HRP@Hf-DBA+H₂O₂. The results are shown in FIG. 6. It can be seen that the HRP@Hf-DBA exhibits the best ability to catalyze generation of ROS from H₂O₂, and can be used for chemodynamic therapy experiments.

Use Example 3 Chemodynamic Therapy for Tumor by HRP@Hf-DBA

1. Cytotoxicity Test

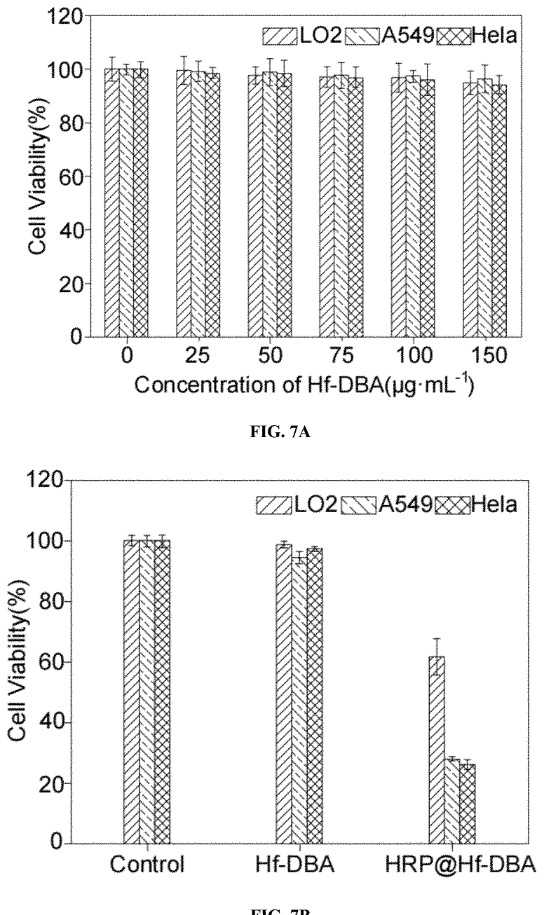

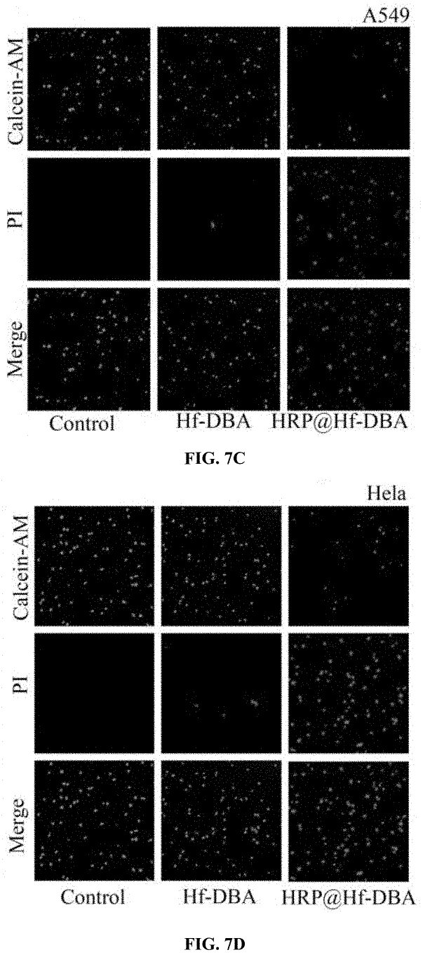

CCK-8, i.e. 2-(2-methoxy-4-nitrophenyl)-3-(4-nitrophenyl)-5-(2,4-disulfophenyl)-2H-tetrazolium sodium salt, could react with substances in living cells to produce formazan, and the amount of formazan is proportional to the number of living cells. Therefore, a light absorption value of CCK-8 at a wavelength of 450 nm measured by an enzyme-linked immunosorbent assay (ELISA) could indirectly reflect the number of living cells. In this use example, the cytotoxicity of HRP@Hf-DBA on human normal hepatocytes (LO2), cervical cancer cells (Hela) and human lung adenocarcinoma cells (A549) was specifically studied by CCK-8. Cell experiments were conducted on an ultra-clean bench after UV light irradiation and alcohol wiping, and cells were cultured in a fully sterilized incubator. Specifically, cells were incubated in a complete medium (containing 10% fetal bovine serum (FBS), 1% dual anti-penicillin-streptomycin, and a DMEM medium). When growing to 80% of a culture dish, the cells were digested with trypsin, and the cells are inoculated into a 96-well cell culture plate for incubation, with a number of cells in each well of about 10,000; after incubating the cells for 24 h, the culture media were replaced with complete media containing Hf-DBA with different concentrations (0 μg/mL, 25 μg/mL, 50 μg/mL, 75 μg/mL, 100 μg/mL, 125 μg/mL, and 150 μg/mL), and the incubation was continued in the incubator for another 24 h. A chemodynamic therapy effect of HRP@Hf-DBA was observed using the same method. Specifically, the complete medium in the 96-well cell culture plate after incubation for 24 h was replaced with a complete medium containing HRP@Hf-DBA, and a cell viability was calculated after incubation in the incubator for 24 h using a CCK-8 method. The results are shown in FIGS. 7A-7B. It can be seen that the cell viabilities of cells added with different concentrations of Hf-DBA are all about 90%, indicating that the Hf-DBA has almost no toxicity and desirable biocompatibility. By comparing three groups of complete medium, Hf-DBA, and HRP@Hf-DBA, it is found that the viability of cells in the HRP@Hf-DBA group are about 27%, indicating a desirable chemodynamic therapy effect.

2. Cell Imaging

To further demonstrate the chemodynamic therapy effect of HRP@Hf-DBA, the tumor cells (A549, Hela) incubated with a PBS buffer solution, Hf-DBA and HRP@Hf-DBA were treated with a Calcein-AM/PI double staining solution for staining. Specifically, tumor cells were inoculated into a 6-well cell culture plate and incubated for 24 h, and then the culture media thereof were replaced with complete media containing Hf-DBA or HRP@Hf-DBA (the Hf-DBA and the HRP@Hf-DBA each had a final concentration of 125 μg/mL) and the tumor cells were incubated therein for another 24 h. All cells were collected, washed 3 times with a PBS buffer solution, and then added into a Calcein-AM/PI double staining solution for staining living and dead cells, After staining for 20 min, cell imaging was conducted using a laser confocal microscope, in which living cells showed green, and dead cells showed red. The results are shown in FIGS. 7C-7D. It can be seen that the tumor cells treated with HRP@Hf-DBA have a higher mortality rate than that of the other two groups, which further proves that the Hf-DBA has desirable biocompatibility and no toxicity, and the HRP@Hf-DBA has a desirable chemodynamic therapy effect on tumor cells.

3. Flow Cytometry of Apoptosis

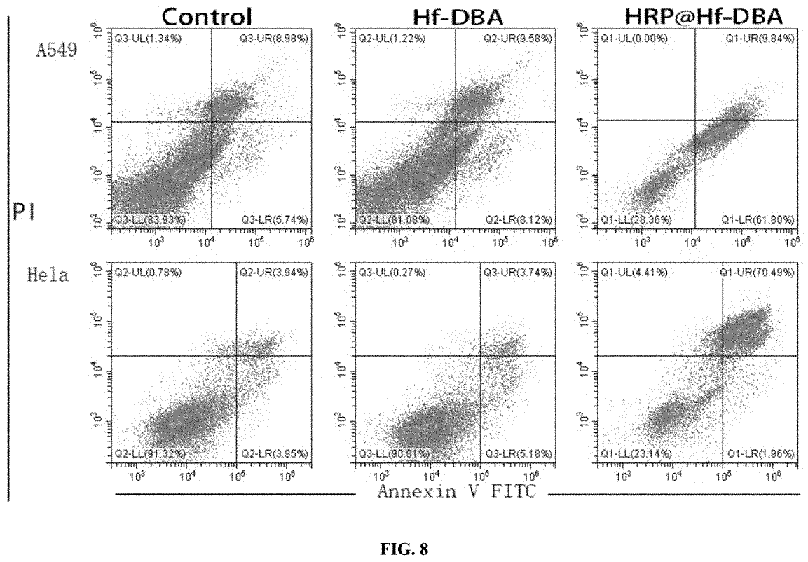

Tumor cells (A549, Hela) were inoculated into a 6-well cell culture plate, with a number of cells in each well of 100,000. The cells were individually incubated for 24 h, and then the culture media thereof were replaced with complete media containing Hf-DBA or HRP@Hf-DBA (the Hf-DBA and the HRP@Hf-DBA each had a final concentration of 125 μg/mL) and the tumor cells were incubated therein for another 24 h. The tumor cells were digested with trypsin; all the tumor cells were collected and washed with a cold PBS buffer solution, centrifuged at 1,000 g for 5 min, and finally resuspended in 195 μL of a 1.0×Binding Buffer to obtain a cell resuspension. 5 μL of Annexin V-FITC and 10 μL of a propidium iodide staining solution were added into 195 μL of the cell resuspension, and mixed to be uniform, and the resulting mixture was incubated at ambient temperature for 15 min in dark. The cells were placed in an ice bath, and subjected to flow cytometry experiments. The results are shown in FIG. 8. It can be seen that the cell viabilities of tumor cells treated with HRP@Hf-DBA are only 28.3% and 23.1%, which are consistent with the results of the previous cell imaging and cytotoxicity test, indicating that the HRP@Hf-DBA has an effect of killing tumor cells.

The above descriptions are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure.

1. A composite nanomaterial based on a metal-organic framework (MOF) material loaded with horseradish peroxidase (HRP), comprising a hafnium-based MOF material and HRP loaded thereon, wherein the hafnium-based MOF material is formed by self-assembly of 2′-amino-1,1′:4,1″-terphenyl-4,4″-dicarboxylic acid and hafnium ions through a coordination bond.

2. The composite nanomaterial based on the MOF material loaded with HRP of claim 1, wherein the composite nanomaterial based on an MOF material loaded with HRP has a loading amount of HRP of 11 wt % to 12 wt %.

3. The composite nanomaterial based on the MOF material loaded with HRP of claim 1, wherein the hafnium-based MOF material has a particle size of 500 nm to 550 nm.

4. A method for preparing the composite nanomaterial based on the MOF material loaded with HRP of claim 1, comprising the following steps:
providing the hafnium-based MOF material, wherein the hafnium-based MOF material is formed by self-assembly of 2′-amino-1,1′:4,1″-terphenyl-4,4″-dicarboxylic acid and the hafnium ions through the coordination bond; and
mixing the hafnium-based MOF material, HRP, and water to obtain a first mixture, and subjecting the first mixture to an incubation to obtain the composite nanomaterial based on an MOF material loaded with HRP.

5. The method of claim 4, wherein each of the hafnium-based MOF material and HRP in the mixture independently has a concentration of 9 mg/mL to 11 mg/mL.

6. The method of claim 4, wherein the incubation is conducted at a temperature of 35° C. to 39° C. for 25 min to 35 min.

7. The method of claim 4, wherein the hafnium-based MOF material is prepared by a process comprising the following steps:
mixing hafnium tetrachloride, 2′-amino-1,1′:4,1″-terphenyl-4,4″-dicarboxylic acid, N,N′-dimethylformamide and acetic acid to obtain a second mixture, and subjecting the second mixture to a solvothermal reaction to obtain the hafnium-based MOF material.

8. A method for inhibiting growth of tumor cells, comprising administrating the composite nanomaterial based on the MOF material loaded with HRP of claim 1 to a subject in need thereof.

9. The method of claim 8, wherein the tumor cells comprise lung adenocarcinoma cells and/or cervical cancer cells.

10. The method of claim 4, wherein the composite nanomaterial based on an MOF material loaded with HRP has a loading amount of HRP of 11 wt % to 12 wt %.

11. The method of claim 4, wherein the hafnium-based MOF material has a particle size of 500 nm to 550 nm.

12. The method of claim 8, wherein the composite nanomaterial based on an MOF material loaded with HRP has a loading amount of HRP of 11 wt % to 12 wt %.

13. The method of claim 8, wherein the hafnium-based MOF material has a particle size of 500 nm to 550 nm.

14. The method of claim 8, wherein the composite nanomaterial based on an MOF material loaded with HRP is prepared by a method comprising the following steps:
providing the hafnium-based MOF material, wherein the hafnium-based MOF material is formed by self-assembly of 2′-amino-1,1′:4,1″-terphenyl-4,4″-dicarboxylic acid and the hafnium ions through the coordination bond; and
mixing the hafnium-based MOF material, HRP, and water to obtain a first mixture, and subjecting the first mixture to an incubation to obtain the composite nanomaterial based on an MOF material loaded with HRP.

15. The method of claim 14, wherein each of the hafnium-based MOF material and HRP in the mixture independently has a concentration of 9 mg/mL to 11 mg/mL.

16. The method of claim 14, wherein the incubation is conducted at a temperature of 35° C. to 39° C. for 25 min to 35 min.

17. The method of claim 14, wherein the hafnium-based MOF material is prepared by a process comprising the following steps:
mixing hafnium tetrachloride, 2′-amino-1,1′:4,1″-terphenyl-4,4″-dicarboxylic acid, N,N′-dimethylformamide and acetic acid to obtain a second mixture, and subjecting the second mixture to a solvothermal reaction to obtain the hafnium-based MOF material.
